(19) United States
(12) Patent Application Publication
(10) Pub. No.: US 2003/0100856 A1
(43) Pub. Date: May 29, 2003

(54) Method and apparatus for alleviating pain

(76) Inventor: Matthew T. Engelbert, Ann Arbor, MI (US)

Correspondence Address: Matthew T. Engelbert, 2424 Antietam Drive, Ann Arbor, MI, 48105-1471, US

(21) Appl. No.: 09/997,328

(22) Filed: Nov. 29, 2001

(51) Int. Cl. A61F 13/00

(52) U.S. Cl. 602/60

(57) ABSTRACT

Pain associated with a wound in a body of a patient caused by contact between a garment worn by the patient and a sensitive portion of the body of the patient proximate the wound may be alleviated by providing at least one spacer for a garment wearable by the patient, wherein the spacer is disposed in a position adjacent the garment wherein, when the patient wears the garment, the spacer does not contact the sensitive portion of the body of the patient proximate the wound, and wherein the spacer has a thickness sufficient to substantially inhibit the garment from contacting the sensitive portion of the body of the patient proximate the wound.

### BACKGROUND

1. Field of the Invention

The present invention relates generally to pain relief and, more particularly, to a method and apparatus for use in alleviating pain such as that associated with a surgical wound or other trauma site on the body of a patient.

2. Related Art

Post-surgical patients (including particularly but without limitation patients who have had hernia surgery or other invasive procedures in the areas of the abdomen or genitals) and other individuals who have sustained wounds or other bodily trauma frequently experience pain that arises not so much from the incision or wound or trauma itself but more from the area of the patient's or other individual's body that surrounds the incision, wound, or trauma. This pain is frequently exacerbated or even triggered by contact between a garment being worn by the patient (e.g., undergarments, shorts, pants, etc.) and a portion of the body of the patient adjacent the incision or other wound.

For example, many surgical procedures involve deep cuts into the abdominal and genital areas and affect large muscles, ligaments, and tendons not only adjacent to but also remote from the area of the actual surgical incision. Obviously the pain and discomfort caused by such deep incisions (or any other wounds) is not limited to the site of the incision or wound. Indeed, areas adjacent to the incision often become numb, and more remote areas outside those numb adjacent areas may become painful and uncomfortable for the patient, particularly when touched by garments being worn by the patient. These latter areas often include the patient's waistline where pants, undergarments, and other clothing impinge directly upon, and thus place direct pressure on, areas of the patient's body that are painfully sensitive to pressure and/or touch.

A number of different approaches have been employed in an effort to alleviate or eliminate that pain and increase the comfort of suffering patients during the recovery and/or healing process with a wide variety of results. Of course, a patient may use over-the-counter or prescription pain medications and/or topical or other local anesthetics, but such measures can produce undesirable side effects and/or drug interactions, particularly for post-surgical patients who may be using other medications in addition to those being taken for pain relief.

Some of these approaches to alleviating pain, such as those disclosed in Mott U.S. Pat. No. 6,080,125, issued Jun. 27, 2000; Mott U.S. Pat. No. 6,270,469, issued Aug. 7, 2001; Tweedle U.S. Pat. No. 5,944,017, issued Aug. 31, 1999; Halderson U.S. Pat. No. 4,134,399, issued Jan. 16, 1979; Kestler U.S. Pat. No. 5,003,972, issued Apr. 2, 1991; and Tweedle U.S. Pat. No. 6,164,279, issued Dec. 26, 2000 are either garments or bandages which seek to provide protection for the exact area of the incision, wound, or trauma itself.

### SUMMARY

The present invention relates to a method and apparatus for alleviating pain that overcomes the foregoing disadvantages previously encountered in connection with prior techniques for alleviating or eliminating pain associated with incisions, wounds, or other sources of bodily pain.

According to one aspect of the present invention, pain associated with a wound in a body of a patient caused by contact between a garment worn by the patient and a sensitive portion of the body of the patient proximate the wound may be alleviated by providing at least one spacer for a garment wearable by the patient, wherein the spacer is disposed in a position adjacent the garment wherein, when the patient wears the garment, the spacer does not contact the sensitive portion of the body of the patient proximate the wound, and wherein the spacer has a thickness sufficient to substantially inhibit the garment from contacting the sensitive portion of the body of the patient proximate the wound.

In accordance with the present invention, the spacer may comprise a conformable or resilient material that substantially conforms to contours of the body of the patient such as, for example, a material selected from the group consisting of gel, silicone, and foam. The spacer may have an adjustable thickness and may be fastenable to a garment to be worn by a patient. More particularly, the spacer may be fastened to the garment by at least one fastener (e.g. a clasp, a clip, hook-and-loop material, a button, a snap, a pin, a staple, a zipper, tape, or adhesive, etc.).

In some embodiments, at least one additional spacer may be provided for the garment, wherein the additional spacer is disposed, for example, in a position adjacent the garment opposite the wound from the at least one spacer so that when the patient wears the garment, the additional spacer does not contact the sensitive portion of the body of the patient proximate the wound, and so that the at least one spacer and the additional spacer together substantially inhibit the garment from contacting the sensitive portion of the body of the patient proximate the wound.

According to another aspect of the present invention, an apparatus may be provided for alleviating pain associated with a wound in a body of a patient, said pain caused (i.e., activated or exacerbated either wholly or partially) by contact between a garment worn by the patient and a sensitive portion of the body of the patient proximate the wound. Such an apparatus may include at least one spacer and a fastener for fastening the at least one spacer in a position adjacent a garment wearable by the patient wherein, when the patient wears the garment, the spacer does not contact the sensitive portion of the body of the patient proximate the wound, and wherein the spacer has a thickness sufficient to substantially inhibit the garment from contacting the sensitive portion of the body of the patient proximate the wound.

According to still another aspect of the present invention, a garment may be provided to be worn by a patient having a wound, wherein a portion of the body of the patient adjacent the wound is sensitive to pressure applied thereto. The garment may at least partially covering the body of a patient having a wound and may include a fabric portion wearable by the patient, at least one spacer having a thickness sufficient to substantially inhibit the garment from contacting the sensitive portion of the body of the patient proximate the wound, and a fastener for fastening the spacer in a position adjacent the fabric portion wherein, when the patient wears the garment, the spacer does not contact the sensitive portion of the body of the patient proximate the wound.

In accordance with still another aspect of the present invention, an apparatus for alleviating pain associated with a wound in the body of a patient, said pain caused by contact between a garment worn by the patient and a sensitive portion of the body of the patient proximate the wound, may include spacing means for spacing a garment worn by the patient away from a sensitive portion of the body of the patient proximate the wound; and fastening means for fastening the spacing means in a position adjacent the garment worn by the patient wherein the spacer does not contact the sensitive portion of the body of the patient proximate the wound. Such an apparatus prevents the garment from contacting the sensitive portion of the body of the patient proximate the wound.

### BRIEF DESCRIPTION OF THE DRAWING

FIG. 1 is a perspective of one exemplary embodiment of a spacer in accordance with the principles of the present invention;

FIG. 2 is an end view of the spacer of FIG. 1 viewed along the lines 2-2 in FIG. 1;

FIG. 3 is a pictorial illustration of a patient wearing a garment over a post-surgical wound, with the spacer of FIG. 1 being used to space the garment away from the body of the patient adjacent the post-surgical wound; and

FIG. 4 is an exploded perspective of the spacer of FIG. 1 in combination with a further spacer element for increasing thickness of the spacer.

### DETAILED DESCRIPTION

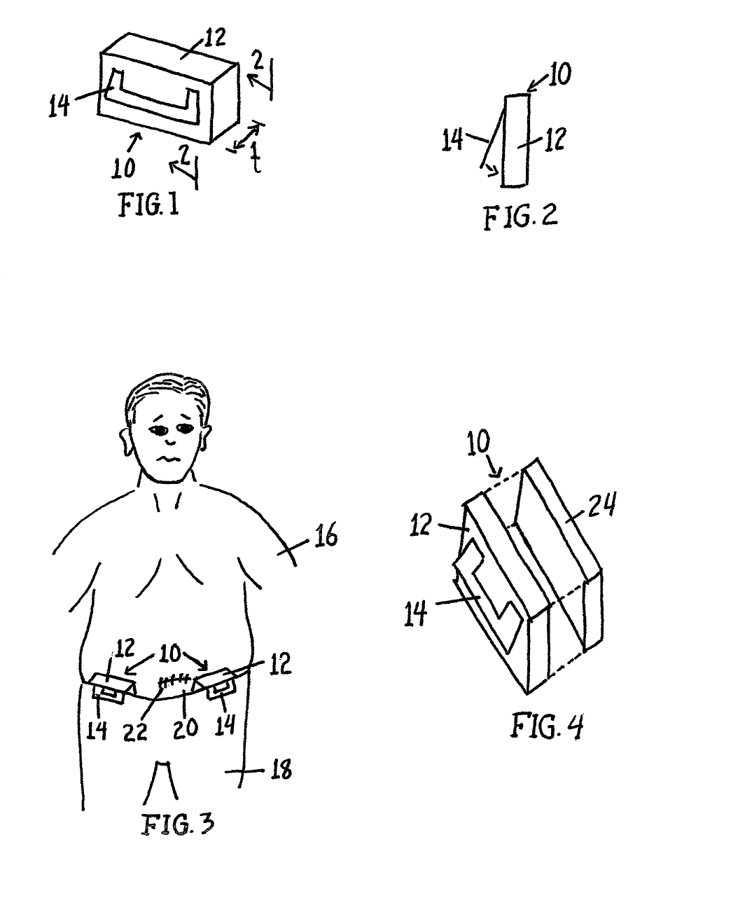

An exemplary embodiment of a spacer 10 in accordance with the principles of the present invention is shown in FIG. 1. The illustrated spacer 10 comprises a generally rectangular member 12 that may be composed of or filled with foam, gel, silicone, or other suitable material which is both conformable to the contours of the body of a patient using the spacer 10 and comfortable for the patient to wear against his or her skin. The spacer 10 also may include a clasp 14 or other suitable fastening means (e.g., a clip, hook-and-loop material, a button, a snap, a pin, a staple, a zipper, tape, adhesive, etc.) affixed or attached to the spacer 10. The clasp 14 or other fastener may be used to be attach the spacer 10 to the inside of undergarments and/or pants on or, more generally, about the waist of the patient in a location where the spacer 10 will not contact the sensitive portion of the body of the patient proximate the wound when the patient wears the garment to which the spacer 10 is attached. The clasp 14 may be composed of metal, plastic or other suitably durable material and may be hinged (as best shown in FIG. 2) or otherwise capable of being easily and securely attached and positioned and easily and securely re-attached and re-positioned on a garment. Of course, adhesive or other fastening means may be used in lieu of the clasp or other fastener 14 to affix the spacer 10 to the garment or even directly to the body of the patient if desired.

The generally rectangular member 12 has a thickness t (illustrated in FIG. 1) which is sufficiently large to substantially inhibit the garment in connection with which the spacer 10 is used from contacting the sensitive portion of the body of the patient proximate the wound. Of course, as will be evident to those of ordinary skill in the art, the thickness t required for any particular spacer 10 may vary depending on the size of the wound, the relative tightness or looseness of the garment, and the density or consistency of the material(s) of which the generally rectangular member 12 is composed. In any event, however, the spacer should be sized in light of the density or consistency of the generally rectangular member 12 so as to ensure that when the generally rectangular member 12 is compressed between the body of the patient and a garment worn by the patient, the generally rectangular member 12 retains sufficient thickness to keep the garment away from the body of the patient adjacent the spacer 10 and, more importantly, the sensitive area adjacent the wound. The spacer 10 then serves to relieve pressure that the garment would otherwise apply to bruised, irritated, swollen and/or otherwise uncomfortable areas of the body of the patient and to create an open area on the body of the patient that is not contacted by clothing being worn by the patient. The spacer 10 thereby provides post-operative comfort for the patient not only at the incision or wound itself over the entire area of the body of the patient surrounding the wound, which may, in many instances, be even more sensitive that the wound itself. By way of example but not limitation, a patient who has undergone abdominal or hernia surgery or a cesarean section may use the spacer to alleviate pain in the vicinity of the surgical wound in the area of the patient's abdomen or genitals, as appropriate.

FIG. 3 depicts a patient 16 wearing a garment 18 with a pair of spacers 10 used to space the garment 18 away from an area 20 of the body of the patient surrounding a wound 22 thereon. As shown, respective generally rectangular members 12 of each of the two spacers 10 are disposed circumferentially adjacent the area 20 on either side thereof. The generally rectangular member 12 of each spacer 12 is fastened to the garment 18 by a respective clasp 14 so that the spacers 10 remain in a generally fixed position relative to the wound 22 and the surrounding area 20 as the patient moves about. As illustrated in FIG. 4, each spacer 10 can be made thicker by attaching to the spacer 10 one or more additional pads 24, which may be composed of foam or any other suitable material conformable to the contours of the body of the patient.

In accordance with the principles of the present invention, the spacer 10 preferably does not cover the incision or wound itself but rather occupies a position adjacent the incision or wound to provide direct protection for the incision or wound while relieving pressure on the greater area surrounding the incision or wound, which area is often bruised, swollen, and painful following surgical incisions, particularly in the areas of the abdomen and genitals. By strategically placing one or more spacers 10 on his undergarments and/or pants, a patient can alleviate or eliminate the discomfort and pressure caused by those garments impinging on sensitive areas of the patients body. As will be appreciated by those of ordinary skill in the art, spacers 10 in accordance with the principles of the present invention may be used in concert with other pain relieving expedients such as those described above, for example.

The foregoing description is for the purpose of teaching those skilled in the art the best mode of carrying out the invention and is to be construed as illustrative only. Numerous modifications and alternative embodiments of the invention will be apparent to those skilled in the art in view of this description, and the details of the disclosed structure may be varied substantially without departing from the spirit of the invention. Accordingly, the exclusive use of all modifications within the scope of the appended claims is reserved.

What is claimed is:

1. A method of alleviating pain associated with a wound in a body of a patient, said pain caused by contact between a garment worn by the patient and a sensitive portion of the body of the patient proximate the wound, the method comprising providing at least one spacer for a garment wearable by the patient, wherein the spacer is disposed in a position adjacent the garment wherein, when the patient wears the garment, the spacer does not contact the sensitive portion of the body of the patient proximate the wound, and wherein the spacer has a thickness sufficient to substantially inhibit the garment from contacting the sensitive portion of the body of the patient proximate the wound.

2. The method of claim 1, wherein the at least one spacer comprises a conformable material.

3. The method of claim 2, wherein the at least one spacer comprises a resilient material that substantially conforms to contours of the body of the patient.

4. The method of claim 2, wherein the at least one spacer comprises a material selected from the group consisting of gel, silicone, and foam.

5. The method of claim 2, wherein the thickness of the at least one spacer is adjustable.

6. The method of claim 1, including fastening the spacer to the garment.

7. The method of claim 6, wherein the spacer is fastened to the garment by at least one fastener selected from the group consisting of a clasp, a clip, hook-and-loop material, a button, a snap, a pin, a staple, a zipper, tape, and adhesive.

8. The method of claim 1, further comprising providing at least one additional spacer for the garment, wherein the additional spacer is disposed in a position adjacent the garment opposite the wound from the at least one spacer wherein, when the patient wears the garment, the additional spacer does not contact the sensitive portion of the body of the patient proximate the wound, and wherein the at least one spacer and the additional spacer together substantially inhibit the garment from contacting the sensitive portion of the body of the patient proximate the wound.

9. An apparatus for alleviating pain associated with a wound in a body of a patient, said pain caused by contact between a garment worn by the patient and a sensitive portion of the body of the patient proximate the wound, the apparatus comprising:
at least one spacer; and
a fastener for the at least one spacer for fastening the at least one spacer in a position adjacent a garment wearable by the patient wherein, when the patient wears the garment, the spacer does not contact the sensitive portion of the body of the patient proximate the wound, and wherein the spacer has a thickness sufficient to substantially inhibit the garment from contacting the sensitive portion of the body of the patient proximate the wound.

10. The apparatus of claim 9, wherein the at least one spacer comprises a conformable material.

11. The apparatus of claim 10, wherein the at least one spacer comprises a resilient material that substantially conforms to contours of the body of the patient.

12. The apparatus of claim 10, wherein the at least one spacer comprises a material selected from the group consisting of gel, silicone, and foam.

13. The apparatus of claim 10, wherein the thickness of the at least one spacer is adjustable.

14. The apparatus of claim 9, wherein the fastener fastens the spacer to the garment.

15. The apparatus of claim 14, wherein the spacer is fastened to the garment by at least one fastener selected from the group consisting of a clasp, a clip, hook-and-loop material, a button, a snap, a pin, a staple, a zipper, tape, and adhesive.

16. The apparatus of claim 9, further comprising at least one additional spacer disposed in a position adjacent the garment opposite the wound from the at least one spacer such that when the patient wears the garment the additional spacer does not contact the sensitive portion of the body of the patient proximate the wound, and wherein the at least one spacer and the additional spacer together substantially inhibit the garment from contacting the sensitive portion of the body of the patient proximate the wound.

17. A garment for at least partially covering the body of a patient having a wound, wherein a portion of the body of the patient adjacent the wound is sensitive to pressure applied thereto, the garment comprising:
a fabric portion wearable by the patient;
at least one spacer having a thickness sufficient to substantially inhibit the garment from contacting the sensitive portion of the body of the patient proximate the wound; and
a fastener for fastening the spacer in a position adjacent the fabric portion wherein, when the patient wears the garment, the spacer does not contact the sensitive portion of the body of the patient proximate the wound.

18. The garment of claim 17, wherein the at least one spacer comprises a conformable material.

19. The garment of claim 18, wherein the at least one spacer comprises a resilient material that substantially conforms to contours of the body of the patient.

20. The garment of claim 18, wherein the at least one spacer comprises a material selected from the group consisting of gel, silicone, and foam.

21. The garment of claim 18, wherein the thickness of the at least one spacer is adjustable.

22. The garment of claim 17, wherein the spacer is fastened to the garment by the fastener.

23. The garment of claim 22, wherein the fastener is selected from the group consisting of a clasp, a clip, hook-and-loop material, a button, a snap, a pin, a staple, a zipper, tape, and adhesive.

24. The garment of claim 17, further comprising at least one additional spacer disposed in a position adjacent the fabric portion opposite the wound from the at least one spacer such that when the patient wears the garment the additional spacer does not contact the sensitive portion of the body of the patient proximate the wound, and wherein the at least one spacer and the additional spacer together substantially inhibit the garment from contacting the sensitive portion of the body of the patient proximate the wound.

25. An apparatus for alleviating pain associated with a wound in the body of a patient, said pain caused by contact between a garment worn by the patient and a sensitive portion of the body of the patient proximate the wound, the apparatus comprising:
spacing means for spacing a garment worn by the patient away from a sensitive portion of the body of the patient proximate the wound; and
fastening means for fastening the spacing means in a position adjacent the garment worn by the patient wherein the spacer does not contact the sensitive portion of the body of the patient proximate the wound;
whereby the garment also does not contact the sensitive portion of the body of the patient proximate the wound.

26. The apparatus of claim 25, wherein the spacing means comprises a conformable material.

27. The apparatus of claim 26, wherein the spacing means comprises a resilient material that substantially conforms to contours of the body of the patient.

28. The apparatus of claim 26, wherein the spacing means comprises a material selected from the group consisting of gel, silicone, and foam.

29. The apparatus of claim 26, wherein the spacing means has an adjustable thickness.

30. The apparatus of claim 25, wherein the fastening means fastens the spacing means to a garment.

31. The apparatus of claim 30, wherein the fastening means is selected from the group consisting of a clasp, a clip, hook-and-loop material, a button, a snap, a pin, a staple, a zipper, tape, and adhesive.

32. The apparatus of claim 25, wherein the spacing means comprises a plurality of spacers disposed in a position adjacent the garment and about the wound such that when the patient wears the garment the spacers do not contact the sensitive portion of the body of the patient proximate the wound, and wherein the plurality of spacers together substantially inhibit the garment from contacting the sensitive portion of the body of the patient proximate the wound.
